(19) United States
(12) Patent Application Publication
(10) Pub. No.: US 2020/0017898 A1
(43) Pub. Date: Jan. 16, 2020

(54) METHOD FOR RAPID IDENTIFICATION OF MICROORGANISMS PRODUCING NUCLEASE ENZYMES

(71) Applicant: TUBITAK, Ankara (TR)

(72) Inventors: Erkan MOZIOGLU, Kocaeli (TR); Sema AKYÜREK, Kocaeli (TR); Simay GÜNDÜZ, Kocaeli (TR); Müslüm AKGÖZ, Kocaeli (TR); Ahmet Ceyhan GÖREN, Kocaeli (TR); Zühtü Tanil KOCAGÖZ, Istanbul (TR)

(73) Assignee: TUBITAK, Ankara (TR)

(21) Appl. No.: 16/493,274

(22) PCT Filed: Mar. 13, 2018
(86) PCT No.: PCT/IB2018/051664

§ 371 Date: Sep. 11, 2019

(30) Foreign Application Priority Data
Mar. 14, 2017 (TR) TR 2017/03901

(51) Int. Cl. C12Q 1/04; C12Q 1/25; C12N 9/22

(52) U.S. Cl. CPC C12Q 1/04; C12N 9/22; C12Q 1/25

(57) ABSTRACT

The invention relates to a method which uses, for the rapid identification of microorganisms producing nuclease enzymes, the quenched DNA/RNA molecules {fluorophore and quencher labelled DNA/RNA molecules) or the gold nanoparticles accumulated on the DNA/RNA molecules.

### TECHNICAL FIELD OF THE INVENTION

The present invention relates to a method for rapid identification of microorganisms producing nuclease enzymes.

The invention more particularly relates to a method for rapid identification of microorganisms producing nuclease enzymes using the quenched DNA/RNA molecules (fluorophore and quencher labelled DNA/RNA molecules) or the gold nanoparticles accumulated on the DNA/RNA molecules.

### PRIOR ART

Today, the identification of nuclease enzymes (DNAse and RNAse) is typically used as in important marker.

To that end, microorganisms are grown in DNA-containing agar media, and then 1N HCl is added to the grown culture and identification can be made taking into account the non-transparency of the medium. These experiments can be conducted at about 18-24 h. In order to make this duration shorter, Devran Gerceker et al. have developed a method that differs from the conventional methods, the so-called DNAse Test Tube method. According to this, whether the organisms grown in a DNA-containing liquid medium produce nuclease enzymes is determined based on the agarose gel image subsequent to culturing. It is claimed that the presence of the bacteria can thus be rapidly identified₍₁₎. Here, however, it is inevitable that the system will not be easy to apply while working on many patient samples since gel running process, which is rather inconvenient, is added to the method as a secondary process.

In the Chinese Patent Application Numbered CN102321759 filed on 25 Aug. 2011 within the state of the art, ‘molecular beacon’ technique is employed for detecting the activity of S1 nucleases.

In the Mexican Patent Application Numbered MXPA05006448 filed on 23 Dec. 2002 within the state of the art, ‘molecular beacon’ technique is employed for detecting the activity of the nucleases cleaving the RNA.

The U.S. Pat. No. 4,719,097 filed on 26 Sep. 1986 within the state of the art, on the other hand, discloses the use of resorufin molecules while detecting the phosphatase enzyme activity.

In the German Patent Application Numbered DE19843873 filed on 25 Sep. 1998 within the state of the art, a method has been developed in which rhodamine 110 molecule is used for the detection of the protease enzyme activity.

In the Patent Application Numbered WO2014207515 filed on 25 Sep. 1998 within the state of the art, a method has been developed in which the nuclease activities can be detected eliminating the need for the quencher molecule.

The Chinese Patent Application Numbered CN101871008 filed on 31 May 2010 within the state of the art discloses the development of a chemical in gel form for use in the identification of fungal acid nucleases. The pH of this chemical, when in liquid form, is 5-6 and it comprises DNA/RNA, zinc, calcium, toluene blue and agar. A method for identifying the nucleases produced by the fungi using this chemical is disclosed.

The aforementioned documents differ from the “method for rapid identification of microorganisms producing nuclease enzymes” according to the invention in terms of content and method.

The U.S. Patent Application Numbered US2003108873 filed on 28 Sep. 2001 within the state of the art relates to the identification of 5′ nucleases and 3′ exonucleases derived from microorganisms on a sequence specificity basis. This method can be employed in the identification and detection of the bacteria or viral diseases in a sample. Particularly, this method differs from the “method for rapid identification of microorganisms producing nuclease enzymes” according to the present invention in that the former is specific to sequences, and thus it is first required to detect the nucleic acid sequences, as well as in terms of the usage purpose and method.

The U.S. Patent Application Numbered US2015211044 filed on 15 Mar. 2015 within the state of the art relates to a method developed for detecting enzymes or microorganisms. In this method, after the microorganisms are concentrated first, they are placed in a solution containing a chromogenic or fluorogenic substrate. In case of the presence of enzyme, it is coupled to chromophores or fluorophores. Therefore, in said invention, the microorganisms are required to be concentrated and kept in a solution which contains substrate. Here, however, in the “method for rapid identification of microorganisms producing nuclease enzymes” according to the present invention, the fluorogenic substrate is directly added to the medium and the principle of an enzyme functioning efficiently in medium conditions is utilized. Hence, the microorganisms can be identified during the growth thereof, based on the presence of enzyme. As a result, the present invention differs from the aforementioned document in that it allows not only the determination of the number of microorganisms but also detecting their resistance against germicidal chemicals.

The lack of the use of quenched DNA/RNA molecules (fluorophore and quencher labelled DNA/RNA molecules) and the gold nanoparticles accumulated on the DNA/RNA molecules in the identification methods of the microorganisms producing nuclease has deemed it necessary to develop the method for rapid identification of microorganisms producing nuclease enzymes according to the invention.

### OBJECTS AND SUMMARY OF THE INVENTION

The object of the present invention is to provide a method of rapidly identifying nuclease enzyme-producing microorganisms in short times expressed as minutes, depending on the bacterial density.

Another object of the present invention is to provide the method of rapid identification of microorganisms producing nuclease enzymes, by adding microorganisms to a microbiological medium having suitable physiological pH values and comprising DNA/RNA, at one terminus of which fluorogenic molecules are present while at the other terminus fluorescence quenching molecules are present, said molecules being coupled to one another.

Another object of the present invention is to provide the method for rapid identification of microorganisms producing nuclease enzymes, by seeding microorganisms in a liquid present in a tube or plate at a suitable pH value such that gold particles will remain positively charged while the DNA/RNA molecules will remain negatively charged.

And another object of the invention is to provide the method for rapid identification of microorganisms producing nuclease enzymes which allows real time monitoring of the same.

The invention relates to a rapid identification method which is used for separating the microorganisms which produce nuclease enzymes and which do not produce nuclease enzymes. Two different types of content can be used in the method. And these are:
Seeding the microorganisms to a liquid or solid microbiological medium containing quenched DNA/RNA molecules (fluorophore and quencher labelled DNA/RNA molecules). The organisms producing nuclease enzymes in this content are detected by means of the fluorescence likely to be observed in the medium.
Seeding the microorganisms to the liquids containing gold nanoparticles and DNA/RNA molecules. In this content, on the other hand, the organisms producing nuclease enzyme are detected by the discoloration likely to be observed in the liquid.

### DETAILED DESCRIPTION OF THE INVENTION

The method for rapid identification of microorganisms producing nuclease enzymes according to the invention is illustrated in the drawings, in which:

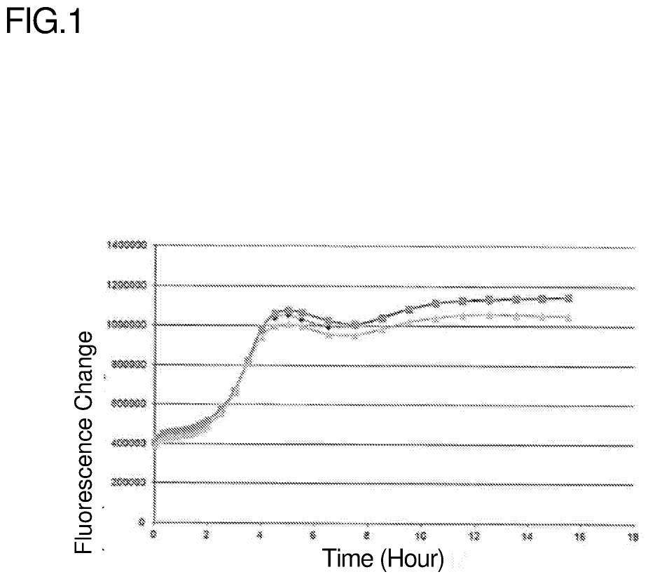

FIG. 1 is the reproduction-dependent change curve of S. pyogenes, which is an organism that produces nuclease enzymes.

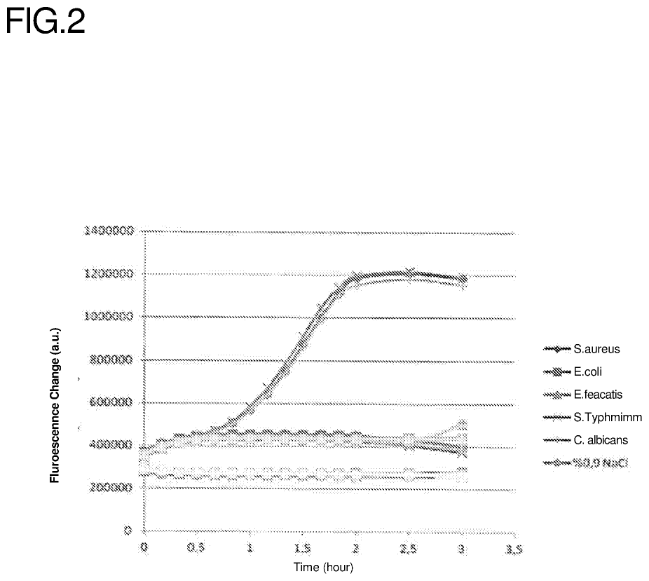

FIG. 2 is the fluorescence change curve of S. aureus, which is an organism that produces nuclease enzymes, dependent on the reproduction of other microorganisms not producing nuclease enzymes.

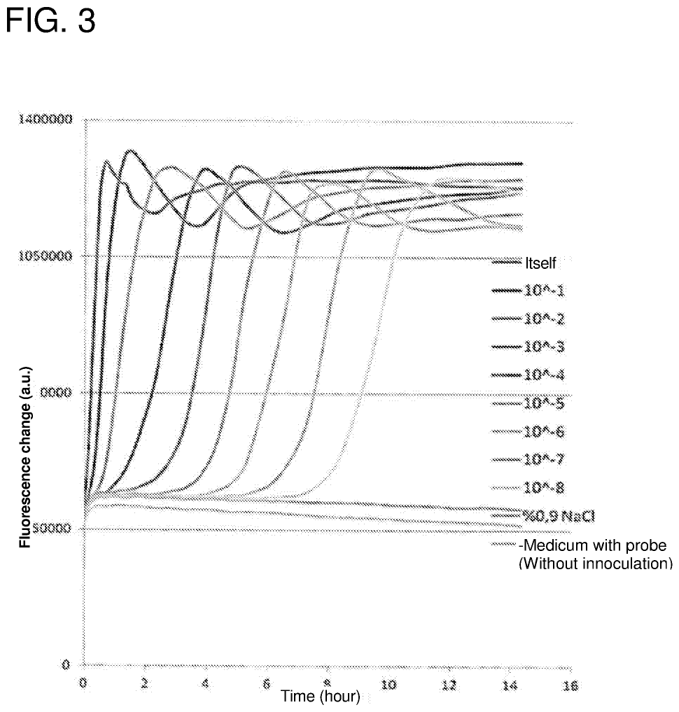

FIG. 3 is the fluorescence change curve of S. aureus, which is an organism that produces nuclease enzymes, dependent on reproduction at different initial concentrations.

The invention is a method for rapid identification of microorganisms producing nuclease enzymes, comprising the steps of:
Coupling to one terminus of the DNA/RNA molecules the fluorogenic/fluorescence molecules (fluorophore), and to the other terminus the fluorescence quenching (quencher) molecules,
Preparing a solution comprising such DNA/RNA molecules and placing the former in the medium,
Adding the microorganisms to be identified to the medium, and
Monitoring the fluorescence signal in the medium by means of an optical apparatus.

The invention is a method for rapid identification of microorganisms producing nuclease enzymes, comprising the steps of:
Preparing a solution consisting of positively charged (positive) gold nanoparticles and negatively charged (negative) DNA/RNA molecules,
Adding the microorganisms to be identified to the thus prepared solution, and
Monitoring the discoloration likely to occur in the solution.

The invention is a method for rapid identification of microorganisms producing nuclease enzymes, wherein a kit comprising a medium having the suitable physiological pH value and in which quenched DNA/RNA (fluorophore and quencher labelled DNA/RNA) molecules are present, is used.

The invention is a method for rapid identification of microorganisms producing nuclease enzymes, wherein a kit comprising a medium having the suitable physiological pH value and in which quenched DNA/RNA (fluorophore and quencher labelled DNA/RNA) molecules are present; suitable tubes and/or plates for fluorescence studies; a shaking or non-shaking incubator to be used in the growth of microorganisms; an optical apparatus for detecting and evaluating the fluorescence signals likely to occur after seeding the microorganisms; and a device with software for the assessment of the signals are used.

The invention is a method for rapid identification of microorganisms producing nuclease enzymes, wherein a kit which consists of positively charged gold nanoparticles and negatively charged DNA/RNA molecules and which comprises a solution having the suitable pH value is used.

The invention is a method for rapid identification of microorganisms producing nuclease enzymes, wherein a kit which consists of positively charged gold nanoparticles and negatively charged DNA/RNA molecules and which comprises a solution having the suitable pH value; tubes and/or plates suitable for discoloration; an optical apparatus for sensing and evaluating the absorption signals associated with the discoloration to occur after seeding the microorganisms; and a device with software used for the assessment of the signals are used.

In an embodiment of the method according to the invention, the fluorescence signal is low in case no microorganism is seeded to a microbiological medium with suitable physiological pH values which comprises the DNA/RNA molecules which at one terminus is labelled with the fluorophore molecules, and with the quencher molecules at the other terminus.

For a rapid identification of the microorganisms producing nuclease enzymes, such microorganisms are seeded to a microbiological medium having suitable physiological pH values and comprising the DNA/RNA molecules which are labelled with fluorophore molecules at one terminus, and with quencher molecules at the other. In case the microorganisms produce nuclease enzyme, the nuclease enzymes of such microorganisms cleave the DNA/RNA molecules. Hence, the quencher molecules and the fluorophore molecules are distanced from one another. An increase in the fluorescence signals is thus observed. Since this change in signal is directly associated with the presence of the nuclease enzymes, and thus of the microorganisms that are capable of producing nuclease enzymes, the identification of such microorganisms dependent on the enzyme production and/or bacterial density can be made in a short time, e.g. 10 minutes to 8 hours.

If the microorganisms do not produce nuclease enzyme, no change in the fluorescence signal is observed due to the absence of cleavage in DNA/RNA molecules.

This fluorescence-based method is not only rapid, but also it allows real time monitoring, which, in turn, makes it possible to perform qualitative (present/absent) identification of the microorganisms producing nuclease enzyme as well as quantitative (bacteria count) identification thereof (FIGS. 1, 2, and 3).

Again, this fluorescence-based method, thanks to the ability of determining the bacteria count, may as well be used in the experiments in which the lowest germicidal (antibiotic/antifungal) concentration of the microorganisms producing nuclease enzymes is detected.

In the method according to the invention, monitoring of the fluorescence signals in the solution is conducted by means of an optical apparatus. The signals are converted and analyzed by the software.

In another embodiment of the method according to the invention, the gold nanoparticles and DNA/RNA molecules are present in a positively charged and negatively charged state, respectively, in the liquid which comprises gold nanoparticles and DNA/RNA molecules and which has a certain pH value. In this case, charge interaction takes place between the negatively charged DNA/RNA molecules and positively charged gold nanoparticles. As a consequence of such interaction, gold nanoparticles accumulate on the DNA/RNA molecules, making the color of the solution change from red to blue.

Because the solution which turned blue in the presence of the DNA/RNA molecules will be cleaved by the nuclease enzymes of the DNA/RNA molecules in the presence of the microorganisms which are placed therein and which are capable of producing nuclease enzymes, the accumulated gold nanoparticles are dispersed in the solution again, making the color of the solution red again. This visible change can be utilized in the rapid identification of the microorganisms producing nuclease enzymes.

The discoloration does not require, apart from the gold nanoparticles, any other chromogenic chemical, e.g. HMRZ-86 ((7R)-7-[2-(aminothiazol-4-1)-(z)-2-(1-carboxy-1-methylethoxyimino)acetamido]-3-(2,4-dinitrostyryl)-3-cephem-4-carboxylic acid trifluoroacetate, E-isomer).

### REFERENCES

(1) Gerceker D, Karasartova D, Elyiirek E, Barkar S, Kiyan M, Ozsan T M, Calgin M K , Sahin F. 2009. A new, simple, rapid test for detection of DNase activity of microorganisms: DNase Tube test. J Gen Appl Microbiol. 55 (4):291-4.

1. The invention is a method for rapid identification of microorganisms producing nuclease enzymes, characterized in that it comprises the steps of:
coupling to one terminus of the DNA/RNA molecules the fluorogenic molecules (fluorophore), and to the other terminus the fluorescence quenching (quencher) molecules,
preparing a solution comprising such DNA/RNA molecules and placing the former in the medium,
seeding the microorganisms to be identified to the medium, and
monitoring the fluorescence signal in the medium by means of an optical apparatus.

2. The invention is a method for rapid identification of microorganisms producing nuclease enzymes, characterized that it comprises the steps of:
preparing a solution consisting of positively charged gold nanoparticles and negatively charged DNA/RNA molecules,
adding the microorganisms to be identified to the solution, and
monitoring the discoloration in the solution.

3. A method for rapid identification of microorganisms producing nuclease enzymes according to claim 1, characterized in that it comprises a kit comprising a medium having the suitable physiological pH value is used.

4. A method for rapid identification of microorganisms producing nuclease enzymes according to claim 3, characterized in that it comprises a kit;
suitable tubes and/or plates for fluorescence studies; a shaking or non-shaking incubator to be used in the growth of microorganisms; an optical apparatus for detecting and evaluating the fluorescence signals likely to occur after seeding the microorganisms; and a device with software for the assessment of the signals are used.

5. A method for rapid identification of microorganisms producing nuclease enzymes according to claim 2, characterized in that it comprises a kit which consists of positively charged gold nanoparticles and negatively charged DNA/RNA molecules and which comprises a solution having the suitable pH value is used.

6. A method for rapid identification of microorganisms producing nuclease enzymes according to claim 5, characterized in that it comprises a kit; tubes and/or plates suitable for discoloration; an optical apparatus for sensing and evaluating the absorption signals associated with the discoloration to occur after seeding the microorganisms; and a device with software used for the assessment of the signals are used.
